(19) United States
(12) Patent Application Publication
(10) Pub. No.: US 2002/0077364 A1
(43) Pub. Date: Jun. 20, 2002

(54) Thyroid hormone formulations

(76) Inventors: Ramaswamy Murari, Hillsborough, NJ (US); Suggy S. Chrai, Cranbury, NJ (US)

Correspondence Address: Allen Bloom, Esq., DECHERT, Princeton Pike Corporate Center, P.O. Box 5218, Princeton, NJ, 08543-5218, US

(21) Appl. No.: 09/900,094

(22) Filed: Jul. 6, 2001

Related U.S. Application Data
(60) Provisional application No. 60/216,275, filed on Jul. 6, 2000.

(51) Int. Cl. A61K 31/198; A61K 9/16; A61K 9/50

(52) U.S. Cl. 514/567; 424/497

(57) ABSTRACT

A method is disclosed for formulating a solid dosage of thyroid hormone, while avoiding instability caused by interaction of the active ingredient with excipients. The thyroid hormone may be levothyroxine sodium or triiodothyronine. The method comprises depositing the active ingredient, preferably electrostatically, as a dry powder substantially free of excipients, onto a pharmaceutically acceptable polymer substrate. Solid pharmaceutical dosage forms also are disclosed.

### BACKGROUND OF THE INVENTION

The present invention relates to improved solid dosage formulations of thyroid hormones. Active physiological thyroid hormones include levothyroxine sodium (the sodium salt of the levo isomer of thyroxine) and triiodothyronine. Thyroid hormone replacement is the therapy of choice for the treatment of primary hypothyroidism, and is also effective for the treatment of secondary hypothyroidism due to pituitary or hypothalamic disease.

Solid pharmaceutical dosages traditionally have included capsules, tablets and other unit dosage forms, each form containing a pharmaceutically or biologically active ingredient and at least one additional “excipient” ingredient. The excipient, which is intended to be a therapeutically inert and non-toxic carrier, may function, for example, as a diluent, binder, lubricant, disintegrant, stabilizer, buffer or preservative.

Thyroid hormone solid dosages historically have suffered from stability problems. For example, from 1994 through 1998, more than 100 million levothyroxine sodium tablets were recalled due to instability, because of an inability to assure adequate potency prior to product expiration. During an approximately one-year period spanning 1999 and 2000, more than 800 million levothyroxine tablets were recalled for similar reasons.

The instability of thyroid hormones, when placed in solid dosage formulations, is believed to be due to drug-excipient interaction. In particular, the formulations are known to be hygroscopic, and degrade rapidly under conditions of high humidity or light and under conditions of high temperature, especially when containing carbohydrate excipients, including lactose, sucrose, dextrose and starch, as well as certain dyes. Thyroid hormones also contains certain functionalities, such as amino and iodo groups, which may be involved in the drug-excipient interactions.

Another problem facing manufacturers of thyroid hormone solid dosage formulations is the need to uniformly mix the thyroid hormone with the various excipients to achieve a satisfactory “content uniformity” of the active ingredient in the formulation matrix. Conventional powder mixing technology is not sufficiently refined to achieve a satisfactorily uniform mix, particularly since the therapeutic dose amount of thyroid hormone is very small. For example, typical daily dosages of levothyroxine sodium range from about 25 micrograms to about 300 micrograms per tablet dosage formulation. Since the ratio of active thyroid hormone to inactive excipients in the tablet matrix ranges from about l-to-450 to about 1-to-5400, the problem of non-uniform mixing can be significant.

In order to fabricate a conventional solid oral dosage form of potent compounds such as levothyroxine, a small particle size, often referred to in the pharmaceutical industry as “micronized powder,” is needed. This permits a more homogeneous blend of the powder with the excipients. However, the aggregate surface area of the particles greatly increases as the median diameter of each particle decreases. With increased surface area of the active ingredient particles comes increased contact with the various excipients, which tends to exacerbate the undesirable drug-excipient interactions. Thus, in order to improve the content uniformity of thyroid hormone solid dosage formulations by decreasing particle size, stability may be further compromised.

Various approaches have been suggested in order to improve the stability of levothyroxine sodium solid dosage forms. For example, U.S. Pat. No. 5,225,204 discloses the use of polyvinylpyrrolidone or Poloxamer as a stabilizing complexing agent for levothyroxine sodium. U.S. Pat. No. 5,635,209 discloses a levothryroxine sodium formulation containing potassium iodide, a disintegrant and a lubricant. U.S. Pat. No. 5,955,105 discloses a solid dosage form comprising a thyroxine drug, a water-soluble glucose polymer and a partially soluble or insoluble cellulose polymer. Published international application WO 99/59551 discloses a formulation containing levothyroxine sodium, gelatin and fillers, free of organic solvent residues. Finally, published international application WO 00/02586 discloses a formulation containing levothyroxine sodium, potassium iodide, microcrystalline cellulose and binding agents, free from anti-oxidizing agents or other adjuvants. Unfortunately, each of these approaches apparently resulted from an onerous search for alternate or additional excipients.

It would be desirable to provide a reliable, stabilized solid dosage formulation of thyroid hormone, which resists degradation by association with commonly used excipients or by exposure to light, heat or humidity.

### SUMMARY OF THE INVENTION

In accordance with the teachings of the present invention, a method is provided for formulating a solid pharmaceutical dosage of thyroid hormone with enhanced stability, which overcomes the disadvantages of the approaches suggested in the prior art.

The method of formulating a solid dosage of thyroid hormone, while avoiding instability caused by interaction of the active ingredient with excipients, comprises depositing the active ingredient, preferably electrostatically, as a dry powder substantially free of excipients, onto a pharmaceutically acceptable polymer substrate. The present invention also includes solid dosage forms prepared according to this method.

It is accordingly an object of the present invention to provide a method for formulating a solid dosage of thyroid hormone with enhanced stability, free of potentially destabilizing excipients, while avoiding the negative consequences previously associated with the use of smaller particle sizes of the active ingredient.

### DETAILED DESCRIPTION OF THE INVENTION

In accordance with the present invention, thyroid hormones can be formulated with increased stability by depositing the active agent, as a dry powder substantially free of excipients, onto a substrate, preferably by an electrostatic deposition process. The active ingredient can be further processed into suitable solid dosage forms.

In the electrostatic deposition process, a cloud or stream of charged particles of the active ingredient is exposed to, or directed towards, a substrate, at the surface of which substrate a pattern of opposite charges has been established. In this fashion, a measured dosage of the active ingredient can be adhered to a substrate without the need for additional carriers, binders or the like. Thus, in a preferred embodiment, thyroid hormone, which normally is unstable when admixed with excipients, is stable when incorporated into a final dosage form using a process of the invention, involving electrostatic deposition.

Suitable means of electrostatic deposition are described in, for example, U.S. Pat. Nos. 5,714,007, 5,846,595 and 6,074,688, the disclosures of which are incorporated by reference herein in their entireties. It also will be appreciated that the active ingredient can be deposited on a pharmaceutical substrate conventionally, such as by using “wet” deposition methods.

Due to the complete elimination of excipients (and, therefore, the elimination of undesirable cross-reactions), increased surface area of the active ingredient is not a liability in the formulations of the present invention. Accordingly, the thyroid hormone can be utilized in any particle size that is amenable to electrostatic deposition and that yields satisfactory content uniformity. A suitable range of particle sizes for the active ingredient is from less than 1 micron (μ) up to about 60μ. In a preferred embodiment, the particle sizes are, on average, less than about 15μ. In a more preferred embodiment, the particle sizes are, on average, less than about 10μ. In the most preferred embodiment, the particle sizes are, on average, less than about 5μ.

The preferred deposition substrate is a “pharmaceutically acceptable” polymer; that is, one that may be introduced safely into the human or animal body, for example, taken orally and digested. Ideally, the polymer has received regulatory approval and is of GRAS (“Generally Regarded As Safe”) status. The substrate polymer, preferably in the form of a film, may either dissolve or otherwise disintegrate subsequent to introduction into the body, for example, subsequent to or upon ingestion, or the polymer may be substantially inert and pass through the body, provided that the dosage form opens or otherwise releases the pharmaceutical substance from the deposit into the patient's body. Suitable materials may include, for example, polymers and copolymers of polyvinyl alcohol, polyvinyl pyrrolidinone, polysaccharide polymers, acrylate polymers, methacrylate polymers, phthalate polymers, polyvinyl acetate, methyl cellulose, carboxymethylcellulose, hydroxyethylcellulose, hydroxypropylcellulose, hydroxypropylmethylcellulose, ethyl cellulose, Eudragits (that is, polymers and copolymers containing methacrylic acid), starch-based polymers, gelatin and the like.

While not wishing to be bound by theory, it is believed that the most useful polymers are those that are substantially unreactive with amino groups or iodo groups in the thyroid hormone molecule. Especially preferred polymers are hydroxypropylcellulose (“HPC”), hydroxypropylmethylcellulose (“HPMC”), ethyl cellulose and combinations thereof.

Preferred dosage forms, as well as additional useful substrate polymers, are disclosed in published international patent application number WO 99/63972, the disclosure of which hereby is incorporated by reference herein in its entirety. For example, a cover film may be applied to encapsulate the electrostatically deposited active ingredient, and the resulting stable “core” may be further processed into dosage forms resembling conventional tablets, capsules, caplets and the like or processed into non-conventional wafers or stamp-like presentations. Each core contains a therapeutic amount of thyroid hormone. Suitable therapeutic amounts generally fall within the range described above.

### EXAMPLES

The following Examples provide the results, respectively, of four studies that were conducted to evaluate the compatibility of levothyroxine sodium with various polymer films. The goal of these studies was to select a suitable polymer film to maximize the stability of levothyroxine sodium for electrostatic deposition, and to develop a dosage form using selected polymer films. In all three studies, samples were stored in individual amber vials with Teflon-lined screw cap closures at 25° C. with 60% Relative Humidity and at 40° C. with 75% Relative Humidity (“RH”).

In each of the following Examples where hydroxypropylmethylcellulose (“HPMC”) was used, the materials are commercially available from Dow Chemical Company, Midland, Michigan in the following grades: The polymer solution of HPMC “E5” at 2% in water has a solution viscosity around 5 cps at 20° C. The polymer solution of HPMC “E50” at 2% in water has a solution viscosity around 50 cps at 20° C.

In each of the Examples where hydroxypropylcellulose (“HPC”) was used, the materials are commercially available from Hercules Chemical Company, Wilmington, Delaware in the following grades: HPC “EFP” has a polymer molecular weight of 80,000. HPC “JFP” has a polymer molecular weight of 140,000.

### Example 1

Levothyroxine samples were made by depositing approximately 250 μg of levothyroxine sodium onto a polymer film formulated with Hydroxypropyl Methylcellulose E50 and Hydroxypropyl Cellulose JFP (50:50) with 10% Polyethylene Glycol 400 (Substrate 1527-69-1). Each sample was sealed using a Branson ultrasonic sealer. The samples were stored at 25° C./60%RH and 40° C./75%RH. As a control, levothyroxine sodium drug substance was stored, without any deposition substrate, in closed amber vials under the same conditions as the samples. Samples were analyzed at 2 weeks and 4 weeks for the presence of degradants by means of a stability-indicating High Performance Liquid Chromatography method. The results are shown in Table 1.

TABLE 1

| 2 weeks | Degradants (Area %) | | | | | | | | | LT (Area %) |
|---|---|---|---|---|---|---|---|---|---|---|
| Average RRT | 0.84 | 0.89 | 1.15 | 1.21 | 1.24 | 1.29 | 1.34 | 1.46 | 1.71 | 1.00 |
| 25° C./60% RH | 0.27 | 0.15 | — | — | — | 0.17 | — | — | 0.49 | 98.7 |
| 40° C./75% RH | 0.20 | 0.16 | — | — | — | 0.57 | 0.32 | 0.10 | 3.55 | 81.6 |

| 4 weeks | Degradants (Area %) | | | | | | LT (Area %) |
|---|---|---|---|---|---|---|---|
| Average RRT | 0.82 | 0.88 | 1.10 | 1.18 | 1.22 | 1.61 | 1.00 |
| 25° C./60% RH | 0.22 | 0.19 | — | 0.34 | — | 0.81 | 98.4 |
| 40° C./75% RH | 0.21 | — | 0.23 | 0.88 | 0.58 | 6.71 | 91.4 |

(Note: RRT refers to a peak's relative retention time, that is, the ratio of its retention time to the retention time of the levothyroxine peak. LT (Area %) is the area percent determination of levothyroxine in the sample, not an assay of levothyroxine versus an external standard.)

### Example 2

The compatibility of levothyroxine sodium with six polymer films was studied. In addition to the film evaluated in Example 1 (1527-69-1), five additional films were evaluated. These consisted of:

1. Substrate 1527-79-1: HPMC E50:HPC JFP (50:50)

2. Substrate 1577-7-1: Ethyl cellulose (“EC”)+5% HPMC E5+35% Triethyl citrate (“TEC”)

3. Substrate 1577-7-3: Ethyl cellulose+5% HPC EFP+35% Triethyl citrate

4. Substrate 1577-6-3: Cellulose acetate phthalate+5% HPMC E5+25% TEC+4% Polysorbate 80

5. Substrate 1577-6-5: Cellulose acetate phthalate+5% HPC EFP+25% TEC+4% Polysorbate 80

Each sample was prepared by depositing approximately 250 μg of levothyroxine sodium on two pieces of polymer substrate, in an amber vial. The vials were sealed with a Teflon-lined screw cap. The samples were stored at 25° C./60%RH and 40° C./75%RH. As a control, levothyroxine sodium drug substance was stored, without any deposition substrate, in closed amber vials under the same conditions as the samples. Samples were analyzed at 2 weeks and 4 weeks for the presence of degradants by means of a stability-indicating High Performance Liquid Chromatography method. The results are shown in Table 2.

TABLE 2
1527-69-1

| 2 weeks | Degradants (Area %) | | | | | | | | | | LT (Area %) |
|---|---|---|---|---|---|---|---|---|---|---|---|
| Average RRT | 0.87 | 0.91 | 0.94 | 1.14 | 1.32 | 1.41 | 1.69 | 1.76 | 1.83 | 2.28 | 1.00 |
| 25° C./60% RH | 0.21 | 0.11 | 0.04 | 0.09 | 0.13 | 0.01 | 0.25 | 0.12 | 0.16 | 0.06 | 98.9 |
| 40° C./75% RH | 0.20 | 0.10 | — | 0.07 | 0.14 | 0.01 | 0.27 | 0.12 | 0.15 | 0.05 | 98.9 |

| 4 weeks | Degradants (Area %) | | | | | | | | | | | | | LT (Area%) |
|---|---|---|---|---|---|---|---|---|---|---|---|---|---|---|
| Average RRT | 0.82 | 0.86 | 0.91 | 1.18 | 1.23 | 1.27 | 1.32 | 1.44 | 1.62 | 1.68 | 1.78 | 1.84 | 2.18 | 1.00 |
| 25° C./60% RH | — | 0.20 | 0.14 | 0.09 | 0.07 | 0.08 | 0.06 | 0.03 | 0.19 | 0.17 | — | 0.15 | 0.05 | 98.8 |
| 40° C./75% RH | 0.01 | 0.20 | 0.13 | 0.10 | 0.05 | 0.14 | 0.02 | 0.03 | — | 0.24 | 0.16 | 0.14 | 0.05 | 98.8 |

1527-79-1

| 2 weeks | Degradants (Area %) | | | | | | | | | | | | LT (Area %) |
|---|---|---|---|---|---|---|---|---|---|---|---|---|---|
| Average RRT | 0.87 | 0.91 | 0.94 | 1.15 | 1.22 | 1.32 | 1.41 | 1.49 | 1.69 | 1.75 | 1.83 | 2.28 | 1.00 |
| 25° C./60% RH | 0.22 | 0.12 | 0.04 | 0.09 | — | 0.12 | 0.01 | — | 0.24 | 0.12 | 0.83 | 0.05 | 98.9 |
| 40° C./75% RH | 0.22 | 0.13 | — | 0.08 | 0.01 | 0.14 | 0.01 | 0.02 | 0.26 | 0.12 | 0.15 | 0.06 | 98.8 |

| 4 weeks | Degradants (Area %) | | | | | | | | | | LT (Area %) |
|---|---|---|---|---|---|---|---|---|---|---|---|
| Average RRT | 0.86 | 0.91 | 1.17 | 1.28 | 1.33 | 1.46 | 1.64 | 1.70 | 1.84 | 2.21 | 1.00 |
| 25° C./60% RH | 0.22 | 0.13 | 0.10 | 0.14 | 0.02 | 0.02 | 0.22 | 0.14 | 0.14 | 0.05 | 98.8 |
| 40° C./75% RH | 0.23 | 0.13 | 0.11 | 0.18 | 0.02 | 0.03 | 0.26 | 0.13 | 0.14 | 0.05 | 98.7 |

1577-7-1

| 2 weeks | Degradants (Area %) | | | | | | | | | LT (Area %) |
|---|---|---|---|---|---|---|---|---|---|---|
| Average RRT | 0.91 | 0.93 | 1.11 | 1.16 | 1.32 | 1.68 | 1.75 | 1.83 | 2.28 | 1.00 |
| 25° C./60% RH | — | — | — | — | — | — | — | — | — | — |
| 40° C./75% RH | 0.04 | 0.02 | 0.09 | 0.0 | 0.15 | 0.31 | 0.12 | 0.14 | 0.04 | 99.1 |

| 4 weeks | Degradants (Area %) | | | | | | | | | | LT (Area %) |
|---|---|---|---|---|---|---|---|---|---|---|---|
| Average RRT | 0.86 | 0.92 | 1.16 | 1.20 | 1.30 | 1.36 | 1.66 | 1.72 | 1.84 | 2.24 | 1.00 |
| 25° C./60% RH | — | — | — | — | — | — | — | — | — | — | — |
| 40° C./75% RH | 0.04 | 0.03 | 0.03 | 0.02 | 0.21 | 0.05 | 0.34 | 0.12 | 0.12 | 0.03 | 99.0 |

1577-7-3

| weeks | Degradants (Area %) | | | | | | | | | | LT (Area %) |
|---|---|---|---|---|---|---|---|---|---|---|---|
| Average RRT | 0.93 | 1.09 | 1.17 | 1.32 | 1.40 | 1.47 | 1.68 | 1.75 | 1.82 | 2.28 | 1.00 |
| 25° C./60% RH | 0.02 | 0.46 | — | 0.13 | — | 0.26 | 0.11 | 0.15 | — | 0.04 | 98.8 |
| 40° C./75% RH | 0.06 | 0.27 | 0.01 | 0.05 | 0.01 | — | 0.31 | 0.11 | 0.14 | 0.03 | 99.0 |

| 4 weeks | Degradants (Area %) | | | | | | | | | | | LT (Area %) |
|---|---|---|---|---|---|---|---|---|---|---|---|---|
| Average RRT | 0.92 | 1.09 | 1.16 | 1.20 | 1.30 | 1.39 | 1.47 | 1.66 | 1.72 | 1.83 | 2.24 | 1.00 |
| 25° C./60% RH | 0.03 | 0.19 | 0.05 | — | 0.14 | 0.03 | 0.02 | 0.26 | 0.09 | 0.14 | 0.06 | 99.1 |
| 40° C./75% RH | 0.04 | 0.15 | 0.03 | 0.02 | 0.18 | — | 0.03 | 0.35 | 0.11 | 0.12 | 0.05 | 99.0 |

1577-6-3

| 2 weeks | Degradants (Area %) | | | | | | | | | | | LT (Area %) |
|---|---|---|---|---|---|---|---|---|---|---|---|---|
| Average RRT | 0.91 | 1.10 | 1.13 | 1.20 | 1.30 | 1.36 | 1.47 | 1.65 | 1.74 | 1.81 | 2.19 | 1.00 |
| 25° C./60% RH | 0.04 | 0.03 | — | — | 0.23 | 0.01 | 3.65 | 0.29 | 0.13 | 0.12 | 0.12 | 95.4 |
| 40° C./75% RH | 0.02 | 0.14 | 0.05 | 0.16 | 0.36 | 0.23 | 24.5 | 5.80 | 0.34 | 0.16 | 5.18 | 63.2 |

| 4 weeks | Degradants (Area %) | | | | | | | | | | | | LT (Area %) |
|---|---|---|---|---|---|---|---|---|---|---|---|---|---|
| Average RRT | 0.86 | 1.10 | 1.16 | 1.20 | 1.29 | 1.34 | 1.46 | 1.65 | 1.71 | 1.81 | 1.84 | 2.23 | 1.00 |
| 25° C./60% RH | 0.13 | — | 0.02 | — | 0.17 | — | 4.53 | — | 0.28 | 0.13 | 0.10 | 0.33 | 94.3 |
| 40° C./75% RH | 0.15 | 0.24 | 0.10 | 0.22 | 0.42 | 0.45 | 27.3 | 0.96 | 0.35 | 0.06 | — | 8.00 | 60.8 |

1577-6-5

| | | |
|---|---|---|
| 2 weeks | Degradants (Area %) | LT (Area %) |

| Average RRT | 0.91 | 0.94 | 1.10 | 1.16 | 1.24 | 1.33 | 1.39 | 1.52 | 1.69 | 1.76 | 1.89 | 2.26 | 3.67 | 3.84 | 1.00 |
|---|---|---|---|---|---|---|---|---|---|---|---|---|---|---|---|
| 25° C./60% RH | 0.04 | — | 0.05 | — | — | 0.21 | 0.01 | 3.05 | 0.27 | 0.12 | 0.13 | 0.14 | — | — | 96.0 |
| 40° C./75% RH | 0.03 | 0.07 | 0.19 | 0.07 | 0.15 | 0.31 | 2.73 | 18.0 | 0.66 | 0.20 | 0.52 | 3.69 | 0.28 | 0.04 | 73.0 |

| | | |
|---|---|---|
| 4 weeks | Degradants (Area %) | LT (Area %) |

| Average RRT | 0.86 | 0.93 | 1.10 | 1.16 | 1.21 | 1.30 | 1.34 | 1.46 | 1.59 | 1.65 | 1.72 | 1.81 | 2.22 | 1.00 |
|---|---|---|---|---|---|---|---|---|---|---|---|---|---|---|
| 25° C./60% RH | 0.15 | 0.06 | — | 0.03 | — | 0.21 | — | 4.77 | — | 0.31 | 0.13 | 0.11 | 0.45 | 93.8 |
| 40° C./75% RH | 0.27 | 0.26 | 0.28 | 0.11 | 0.30 | 0.43 | 0.59 | 33.8 | 0.26 | 1.14 | 0.36 | 0.06 | 10.6 | 51.3 |

It can be seen from the data above that there was significant loss of active ingredient with the cellulose acetate phthalate films, when stored at both 25° C./60%RH and 40° C./75%RU. The films containing this material were dropped from further consideration. The remaining four films (the HPMC/HPC and EC combination films) showed potential for further development; in each case, loss of active ingredient was less than 5%. Thus, a third compatibility study was undertaken with these films to confirm the results of this study.

### Example 3

The compatibility of levothyroxine sodium with six polymer films was studied. Included in the study were the four HIPMC/HPC and ethyl cellulose films studied in Example 2 (Substrates 1527-79-1, 1577-7-1, 1577-7-3 and 1527-69-1). Film 1527-69-1 was included because of the somewhat different results obtained in Examples 1 and 2, respectively. The remaining three films were included to confirm the results of Example 2. Also included were two additional films made from individual polymer components; a film comprised of HPC JFP (Substrate 1527-84-1) and a film comprised of HPMC E50 (Substrate 1501-56-3). In this study, the quantities of drug and of polymer film were increased, while maintaining the approximately 1:14 drug-to-film ratio of Examples 1 and 2. Once again the samples were stored in amber vials with Teflon-lined screw cap closures at 25° C./60%RH and 40° C./75%RH. Drug substance stored in closed amber vials, without any deposition substrate, served as a control for this study. Samples were analyzed at 2 weeks and 6 weeks for the presence of degradants by means of a stability-indicating High Performance Liquid Chromatography method. The results of this study are shown in Table 3.

TABLE 3
1527-79-1

| 2 weeks | Degradants (Area %) | | | | | | | | | | | | | | LT (Area %) |
|---|---|---|---|---|---|---|---|---|---|---|---|---|---|---|---|
| Average RRT | 0.88 | 0.92 | 0.94 | 1.16 | 1.23 | 1.34 | 1.42 | 1.49 | 1.51 | 1.70 | 1.77 | 1.85 | 2.30 | 2.41 | 1.00 |
| 25° C./60% RH | 0.20 | 0.10 | — | 0.09 | — | 0.23 | 0.02 | — | 0.02 | 0.26 | 0.13 | 0.16 | 0.09 | 0.01 | 98.7 |
| 40° C./75% RH | 0.20 | 0.10 | 0.03 | 0.12 | 0.03 | 0.32 | 0.04 | 0.01 | 0.02 | 0.28 | 0.13 | 0.15 | 0.09 | 0.02 | 98.5 |

| 6 weeks | Degradants (Area %) | | | | | | | LT (Area %) |
|---|---|---|---|---|---|---|---|---|
| Average RRT | 0.88 | 0.92 | 1.14 | 1.34 | 1.70 | 1.82 | 2.30 | 1.00 |
| 25° C./60% RH | 0.18 | 0.11 | 0.14 | 0.22 | 0.26 | 0.29 | 0.07 | 98.7 |
| 40° C./75% RH | 0.17 | 0.09 | 0.12 | 0.25 | 0.30 | 0.28 | 0.07 | 98.8 |

1527-84-1

| 2 weeks | Degradants (Area %) | | | | | | | | | | | | | | LT (Area %) |
|---|---|---|---|---|---|---|---|---|---|---|---|---|---|---|---|
| Average RRT | 0.88 | 0.92 | 0.95 | 1.16 | 1.24 | 1.32 | 1.38 | 1.50 | 1.51 | 1.70 | 1.77 | 1.85 | 2.30 | 2.41 | 1.00 |
| 25° C./60% RH | 0.18 | 0.07 | — | 0.08 | — | 0.23 | 0.03 | — | 0.02 | 0.26 | 0.13 | 0.15 | 0.09 | 0.01 | 98.7 |
| 40° C./75% RH | 0.19 | 0.07 | 0.04 | 0.11 | 0.05 | 0.15 | 0.16 | 0.03 | 0.02 | 0.28 | 0.13 | 0.15 | 0.09 | 0.01 | 98.6 |

| 6 weeks | Degradants (Area %) | | | | | | | | | LT (Area %) |
|---|---|---|---|---|---|---|---|---|---|---|
| Average RRT | 0.88 | 0.92 | 0.95 | 1.14 | 1.34 | 1.52 | 1.70 | 1.82 | 2.30 | 1.00 |
| 25° C./60% RH | 0.18 | 0.10 | — | 0.17 | 0.25 | — | 0.27 | 0.28 | 0.07 | 98.7 |
| 40° C./75% RH | 0.18 | 0.09 | 0.04 | 0.11 | 0.27 | 0.01 | 0.32 | 0.27 | 0.07 | 98.7 |

1501-56-3

| 2 weeks | Degradants (Area %) | | | | | | | | | | | | | LT (Area %) |
|---|---|---|---|---|---|---|---|---|---|---|---|---|---|---|
| Average RRT | 0.88 | 0.92 | 1.16 | 1.23 | 1.34 | 1.42 | 1.49 | 1.51 | 1.70 | 1.77 | 1.85 | 2.29 | 2.41 | 1.00 |
| 25° C./60% RH | 0.20 | 0.11 | 0.14 | — | 0.27 | 0.03 | — | 0.02 | 0.28 | 0.12 | 0.16 | 0.09 | 0.02 | 98.6 |
| 40° C./75% RH | 0.20 | 0.11 | 0.11 | 0.04 | 0.29 | 0.03 | 0.01 | 0.02 | 0.31 | 0.13 | 0.16 | 0.09 | 0.01 | 98.5 |

| 6 weeks | Degradants (Area %) | | | | | | | LT (Area %) |
|---|---|---|---|---|---|---|---|---|
| Average RRT | 0.88 | 0.92 | 1.14 | 1.34 | 1.70 | 1.83 | 2.30 | 1.00 |
| 25° C./60% RH | 0.18 | 0.11 | 0.20 | 0.25 | 0.33 | 0.25 | 0.07 | 98.6 |
| 40° C./75% RH | 0.19 | 0.10 | 0.13 | 0.25 | 0.32 | 0.26 | 0.07 | 98.7 |

1577-7-1

| 2 weeks | Degradants (Area %) | | | | | | | | | | | | | | LT (Area %) |
|---|---|---|---|---|---|---|---|---|---|---|---|---|---|---|---|
| Average RRT | 0.93 | 1.18 | 1.23 | 1.33 | 1.43 | 1.48 | 1.50 | 1.69 | 1.76 | 1.84 | 1.93 | 2.13 | 2.28 | 2.37 | 1.00 |
| 25° C./60% RH | 0.08 | 0.07 | — | 0.25 | 0.02 | — | — | 0.24 | 0.08 | 0.12 | — | 0.05 | 0.04 | 0.02 | 99.0 |
| 40° C./75% RH | 0.07 | 0.07 | 0.03 | 0.32 | 0.03 | 0.01 | 0.02 | 0.31 | 0.08 | 0.11 | 0.01 | 0.05 | 0.04 | 0.02 | 98.8 |

| 6 weeks | Degradants (Area %) | | | | | | | LT (Area %) |
|---|---|---|---|---|---|---|---|---|
| Average RRT | 0.91 | 1.21 | 1.32 | 1.68 | 1.80 | 2.12 | 2.27 | 1.00 |
| 25° C./60% RH | 0.02 | — | 0.29 | 0.33 | 0.25 | 0.11 | — | 99.0 |
| 40° C./75% RH | 0.02 | 0.01 | 0.34 | 0.54 | 0.20 | 0.09 | 0.07 | 98.7 |

1577-7-3

| 2 weeks | Degradants (Area %) | | | | | | | | | | | | | | LT (Area %) |
|---|---|---|---|---|---|---|---|---|---|---|---|---|---|---|---|
| Average RRT | 0.91 | 0.96 | 1.17 | 1.23 | 1.32 | 1.39 | 1.49 | 1.69 | 1.75 | 1.83 | 1.93 | 2.13 | 2.28 | 2.37 | 1.00 |
| 25° C./60% RH | 0.17 | — | 0.04 | 0.01 | 0.26 | 0.02 | 0.02 | 0.24 | 0.06 | 0.10 | — | 0.06 | 0.03 | 0.04 | 98.9 |
| 40° C./75% RH | 0.07 | 0.04 | 0.07 | 0.03 | 0.32 | 0.03 | 0.03 | 0.31 | 0.08 | 0.11 | 0.01 | 0.05 | 0.04 | 0.02 | 98.8 |

| 6 weeks | Degradants (Area %) | | | | | | | | | | LT (Area %) |
|---|---|---|---|---|---|---|---|---|---|---|---|
| Average RRT | 0.96 | 1.13 | 1.21 | 1.32 | 1.45 | 1.68 | 1.79 | 2.11 | 2.25 | 2.35 | 1.00 |
| 25° C./60% RH | — | — | — | 0.31 | — | 0.35 | 0.24 | 0.19 | 0.02 | 0.18 | 98.7 |
| 40° C./75% RH | 0.03 | 0.07 | 0.01 | 0.47 | 0.03 | 0.86 | 0.18 | — | 0.03 | 0.16 | 98.0 |

1527-69-1

| 2 weeks | Degradants (Area %) | | | | | | | | | | | | | |
|---|---|---|---|---|---|---|---|---|---|---|---|---|---|---|
| Average RRT | 0.79 | 0.88 | 0.92 | 0.95 | 1.16 | 1.20 | 1.23 | 1.33 | 1.42 | 1.51 | 1.70 | 1.77 | 1.85 | 1.93 |
| 25° C./60% RH | 0.01 | 0.22 | 0.11 | 0.04 | 0.19 | — | — | 0.30 | 0.04 | 0.03 | 0.29 | 0.13 | 0.16 | 0.05 |
| 40° C./75% RH | — | 0.21 | 0.09 | — | 0.06 | 0.01 | 0.01 | 0.11 | 0.03 | 0.05 | 0.62 | 0.15 | 0.14 | — |

| | Degradants (Area %) | | | | LT (Area %) |
|---|---|---|---|---|---|
| Average RRT | 2.30 | 2.41 | 3.83 | 4.46 | 1.00 |
| 25° C./60% RH | 0.09 | 0.01 | — | — | 98.4 |
| 40° C./75% RH | 0.11 | 0.02 | 0.03 | 0.01 | 98.0 |

| 6 weeks | Degradants (Area %) | | | | | | | | | | | | | | LT (Area %) |
|---|---|---|---|---|---|---|---|---|---|---|---|---|---|---|---|
| Average RRT | 0.88 | 0.92 | 0.95 | 1.14 | 1.23 | 1.34 | 1.51 | 1.70 | 1.82 | 2.29 | 2.68 | 2.94 | 3.84 | 4.44 | 1.00 |
| 25° C./60% RH | 0.19 | 0.12 | — | 0.10 | — | 0.30 | 0.03 | 0.44 | 0.26 | 0.07 | — | — | 3.84 | 4.46 | 98.3 |
| 40° C./75% RH | 0.18 | 0.09 | 0.07 | — | 0.25 | 1.25 | 0.38 | 2.03 | 0.17 | 0.12 | 0.06 | 0.06 | 0.06 | 0.05 | 95.3 |

These results indicate that certain polymers were associated with an undesirable loss of active ingredient. However, five of the eight polymer film formulations were associated with a loss of not more than 2% of the active ingredient under stress conditions. Thus, it is apparent that polymers having a high degree of compatibility with thyroid hormone (that is, which result in negligible loss of the active ingredient) can be identified readily from the routine screening of polymers that are conventional for pharmaceutical use.

### Example 4

A long-term stability study was performed with respect to Substrate 1527-79-1, which had demonstrated good performance over a four-week period in Example 3.

This follow-up study, employing the same conditions as in Example 3, was carried out for 26 weeks. Two different particle-size lots of active ingredient (designated 90206A and 90206B) were used.

The results of this study are shown in Table 4, which demonstrates continued stability of the active ingredient after 8, 13 and 26 weeks.

TABLE 4
Study B

| 4 Weeks | 1000 ug per sample Lot 90206A | | | | | | | | | | | | | | |
|---|---|---|---|---|---|---|---|---|---|---|---|---|---|---|---|
| Average RRT | 0 57 | 0 63 | 0 80 | 0 92 | 0 94 | 1 16 | 1.23 | 1 34 | 1 42 | 1 49 | 1 51 | 1 70 | 1 77 | 1 85 | 2 30 |
| 25/60 | 0 10 | 0 20 | 0 20 | 0 10 | | | 0 10 | 0 10 | | | | 0.20 | 0 10 | 0 10 | |
| 40/75 | 0 10 | 0 30 | 0 20 | 0 10 | | | | | | | | 0.20 | 0 20 | | |

Study B

| 4 Weeks | 1000 ug per sample Lot 90206A | | | | | | | | | | | | |
|---|---|---|---|---|---|---|---|---|---|---|---|---|---|
| Average RRT | 2 41 | 2 54 | 2.69 | 2.95 | 3 11 | 3 18 | 3 45 | 3 60 | 4 10 | 4.31 | 4 34 | 4 40 | 4.51 |
| 25/60 | | | 0 10 | | | | | | | 0 10 | | | |
| 40/75 | | | 0 10 | | | | | | | 0.10 | | | |

| Study B | | | | | | |
|---|---|---|---|---|---|---|
| 4 Weeks | 1000 ug per sample Lot 90206A | | | | | LT Area % |
| Average RRT | 4 68 | 4 84 | 5 00 | 5 10 | 5 40 | LT Area % |
| 25/60 | | | 0.10 | | 0 20 | 98 9 |
| 40/75 | | | 0.10 | 0 10 | 0 20 | 98.5 |

Study B

| 8 Weeks | 1000 ug per sample Lot 90206A | | | | | | | | | | | | | | | |
|---|---|---|---|---|---|---|---|---|---|---|---|---|---|---|---|---|
| Average RRT | 0 57 | 0 63 | 0 88 | 0 92 | 0 94 | 1 16 | 1.23 | 1 34 | 1 42 | 1 49 | 1 51 | 1 70 | 1 77 | 1 85 | 2 19 | 2 30 |
| 25/60 | | 0 10 | 0 20 | 0 10 | 0 10 | | 0 10 | | | | | | 0 10 | | | |
| 40/75 | 0 30 | 0 20 | 0 20 | 0 10 | | | | 0.10 | | | 0 10 | | 0 10 | | 0 20 | |

Study B

| 8 Weeks | 1000 ug per sample Lot 90206A | | | | | | | | | | | | |
|---|---|---|---|---|---|---|---|---|---|---|---|---|---|
| Average RRT | 2 41 | 2 54 | 2.69 | 2.95 | 3 11 | 3 18 | 3 45 | 3 60 | 4 10 | 4.31 | 4 34 | 4 40 | 4.51 |
| 25/60 | | | | | | | | | | 0.10 | | | |
| 40/75 | | 0 10 | | 0 10 | | 0 20 | | | 0 20 | 0 10 | 0 10 | 0.10 | 0.10 |

| Study B | | | | | | |
|---|---|---|---|---|---|---|
| 8 Weeks | 1000 ug per sample Lot 90206A | | | | | |
| Average RRT | 4 68 | 4 84 | 5 00 | 5 10 | 5 40 | |
| 25/60 | | | 0 10 | | 0 20 | 99 1 |
| 40/75 | | | | | | 98 4 |

Study B

| 13 Weeks | 1000 ug per sample Lot 90206A | | | | | | | | | | | | | | | |
|---|---|---|---|---|---|---|---|---|---|---|---|---|---|---|---|---|
| Average RRT | 0 57 | 0 63 | 0 88 | 0 92 | 0 94 | 1 16 | 1.23 | 1 34 | 1 42 | 1 49 | 1 51 | 1 70 | 1 77 | 1 85 | 2 19 | 2 30 |
| 25/60 | 0 10 | 0 10 | 0 20 | | | | | 0 10 | | | 0 10 | | 0 10 | | 0 10 | 0 10 |
| 40/75 | 0 10 | 0 20 | 0 20 | 0 10 | | | | 0 10 | | | 0 10 | | 0 30 | 0 10 | 0 10 | |

Study B

| 13 Weeks | 1000 ug per sample Lot 90206A | | | | | | | | | | | | | |
|---|---|---|---|---|---|---|---|---|---|---|---|---|---|---|
| Average RRT | 2 41 | 2 54 | 2.69 | 2.95 | 3 11 | 3 18 | | 3 45 | 3 60 | 4 20 | 4.31 | 4 34 | 4 40 | 4.51 |
| 25/60 | | | | 0.10 | | 0 10 | 0.20 | | | 0 10 | 0 10 | | | |
| 40/75 | | | 0.10 | 0 10 | 0 20 | | | | | 0 10 | | | | 0.10 |

| Study B | | | | | | |
|---|---|---|---|---|---|---|
| 13 Weeks | 1000 ug per sample Lot 90206A | | | | | |
| Average RRT | 4 68 | 4 84 | 5 00 | 5 10 | 5 40 | |
| 25/60 | | | 0 10 | 0 20 | | 98 4 |
| 40/75 | 0 10 | | 0 20 | 0 60 | | 96 7 |

Study B

| 13 Weeks | 1000 ug per sample Lot 90206B | | | | | | | | | | | | | | | |
|---|---|---|---|---|---|---|---|---|---|---|---|---|---|---|---|---|
| Average RRT | 0 57 | 0 63 | 0 88 | 0 92 | 0 94 | 1 16 | 1.23 | 1 34 | 1 42 | 1 49 | 1 51 | 1 70 | 1 77 | 1 85 | 2 19 | 2 30 |
| 25/60 | | 0 10 | 0 20 | | | | | | | | | | 0 10 | | | |
| 40/75 | | | 0 20 | | | | | | | | 0 10 | | 0 20 | 0 10 | | |

Study B

| 13 Weeks | 1000 ug per sample Lot 90206B | | | | | | | | | | | | | |
|---|---|---|---|---|---|---|---|---|---|---|---|---|---|---|
| Average RRT | 2 41 | 2 54 | 2.69 | 2.95 | 3 11 | 3 18 | 3 28 | 3 45 | 3 60 | 4 10 | 4.31 | 4 34 | 4 40 | 4.51 |
| 25/60 | | | 0 10 | 0 10 | | | 0 10 | | | 0 10 | 0 20 | | | |
| 40/75 | | | 0 10 | 0 20 | | | 0 30 | | | 0 10 | | 0 20 | | |

| Study B | | | | | | |
|---|---|---|---|---|---|---|
| 13 Weeks | 1000 ug per sample Lot 90206B | | | | | |
| Average RRT | 4 68 | 4 84 | 5 00 | 5 10 | 5 40 | |
| 25/60 | | | | | | 98 7 |
| 40/75 | | 0 10 | 0 10 | | | 98 1 |

Study B

| 26 Weeks | 1000 ug per sample Lot 90206A | | | | | | | | | | | | | | | |
|---|---|---|---|---|---|---|---|---|---|---|---|---|---|---|---|---|
| Average RRT | 0 57 | 0 63 | 0 88 | 0 92 | 0 94 | 1 16 | 1.23 | 1 34 | 1 42 | 1 49 | 1 51 | 1 70 | 1 77 | 1 85 | 2 19 | 2 30 |
| 25/60 | 0 20 | 0 20 | 0 10 | | | | | 0 10 | | | 0 10 | 0 20 | 0 10 | | | 0 10 |
| 40/75 | 0 20 | 0 20 | | 0 10 | 0 10 | 0 20 | | 0 10 | 0 10 | | 0 40 | 0 20 | | 0 10 | 0 10 | |

Study B

| 26 Weeks | 1000 ug per sample Lot 90206A | | | | | | | | | | | | | | |
|---|---|---|---|---|---|---|---|---|---|---|---|---|---|---|---|
| Average RRT | 2 41 | 2 54 | 2.69 | 2.95 | 3 11 | 3 18 | | 3 45 | 3 50 | 3 60 | 4 10 | 4.31 | 4 34 | 4 40 | 4.51 |
| 25/60 | | 0 10 | 0 10 | | | 0 30 | | | | | | | | | |
| 40/75 | 0 10 | 0 30 | 0 60 | | 0 40 | 0 20 | 0 10 | 0 10 | 0 10 | 0 10 | | | | | 1 00 |

| Study B | | | | | | |
|---|---|---|---|---|---|---|
| 26 Weeks | 1000 ug per sample Lot 90206A | | | | | |
| Average RRT | 4 68 | 4 84 | 5 00 | 5 10 | 5 40 | |
| 25/60 | | | | | | 98 2 |
| 40/75 | | | | | | 94 9 |

Study B

| 26 Weeks | 1000 ug per sample Lot 90206B | | | | | | | | | | | | | | | |
|---|---|---|---|---|---|---|---|---|---|---|---|---|---|---|---|---|
| Average RRT | 0 57 | 0 63 | 0 88 | 0 92 | 0 94 | 1 16 | 1.23 | 1 34 | 1 42 | 1 49 | 1 51 | 1 70 | 1 77 | 1 85 | 2 19 | 2 30 |
| 25/60 | | | 0 20 | | | | 0 10 | | | | 0 10 | | | | | 0 10 |
| 40/75 | | | 0 30 | | | 0 20 | | | 0 20 | | 0 20 | 0 10 | | 0 10 | | |

Study B

| 26 Weeks | 1000 ug per sample Lot 90206B | | | | | | | | | | | | | | | |
|---|---|---|---|---|---|---|---|---|---|---|---|---|---|---|---|---|
| Average RRT | 2 41 | 2 50 | 2 54 | 2.69 | 2.95 | 3 11 | 3 18 | | 3 45 | 3 50 | 3 60 | 4 10 | 4.31 | 4 34 | 4 40 | 4.51 |
| 25/60 | | | 0 10 | 0 10 | | | 0 80 | | 0 10 | | | | | | | |
| 40/75 | 0 10 | 0 30 | 0 50 | | 0 30 | | 0 10 | 0 20 | 0 10 | | 0 10 | | | | | 0 80 |

| Study B | | | | | | |
|---|---|---|---|---|---|---|
| 26 Weeks | 1000 ug per sample Lot 90206B | | | | | |
| Average RRT | 4 68 | 4 84 | 5 00 | 5 10 | 5 40 | |
| 25/60 | | | | | | 98 1 |
| 40/75 | | | | | | 96 4 |

Although the present invention has been described with particular reference to certain preferred embodiments thereof, variations and modifications of the present invention can be effected within the spirit and scope of the following claims.

We claim:

1. A method of formulating a solid dosage of thyroid hormone, while avoiding instability caused by interaction of the active ingredient with excipients, comprising depositing the active ingredient, as a dry powder substantially free of excipients, onto a pharmaceutically acceptable polymer substrate.

2. The method of claim 1, wherein the depositing is performed electrostatically.

3. The method of claim 1, wherein the thyroid hormone is levothyroxine sodium or triiodothyronine.

4. The method of claim 1, wherein the polymer has received regulatory approval and is of GRAS status.

5. The method of claim 4, wherein the polymer is selected from the group consisting of polyvinyl alcohol, polyvinyl pyrrolidinone, polysaccharide polymers, acrylate polymers, methacrylate polymers, phthalate polymers, polyvinyl acetate, methyl cellulose, carboxymethylcellulose, hydroxyethylcellulose, hydroxypropylcellulose, hydroxypropylmethylcellulose, ethyl cellulose, Eudragits, starch-based polymers, gelatin, and combinations thereof.

6. The method of claim 4, wherein the polymer is substantially unreactive with an amino group or iodo group in the thyroid hormone molecule.

7. The method of claim 6, wherein the polymer is selected from the group consisting of hydroxypropylcellulose, hydroxypropylmethylcellulose, ethyl cellulose and combinations thereof.

8. An improved solid pharmaceutical dosage formulation, comprising a therapeutic amount of thyroid hormone, electrostatically deposited on a pharmaceutically acceptable polymer substrate as a dry powder substantially free of excipients, wherein the average powder particle size is less than about 15μ.

9. The formulation of claim 8, wherein the thyroid hormone is levothyroxine sodium or triiodothyronine.

10. The formulation of claim 8, wherein the average powder particle size is less than about 10μ.

11. The formulation of claim 8, wherein the average powder particle size is less than about 5μ.

12. The formulation of claim 8, wherein the polymer is substantially unreactive with an amino group or iodo group in the thyroid hormone molecule.

13. The method of claim 2, further comprising:
(a) applying a cover film to encapsulate the electrostatically deposited active ingredient, so as to form a stable core; and
(b) further processing the stable core into a dosage form resembling a tablet, capsule, caplet, wafer or stamp-like presentation.
